(19) United States
(12) Patent Application Publication
(10) Pub. No.: US 2010/0150976 A1
(43) Pub. Date: Jun. 17, 2010

(54) BIOLOGICALLY DEGRADABLE MULTILAYER SYSTEM

(76) Inventors: Iris Schnitzler, Bonn (DE); Christian Hausen, Vettelschoss (DE); Michael Roreger, Neuwied (DE); Dierk Schumacher, Melsungen (DE)

Correspondence Address: FROMMER LAWRENCE & HAUG, 745 FIFTH AVENUE- 10TH FL., NEW YORK, NY, 10151, US

(21) Appl. No.: 12/594,393

(22) PCT Filed: Apr. 2, 2008
(86) PCT No.: PCT/EP2008/002620

§ 371 Date: Oct. 2, 2009

(30) Foreign Application Priority Data
Apr. 4, 2007 (DE) 10 2007 016 684.4

(51) Int. Cl. A61K 8/02; A61K 9/70; A01N 25/34; A61K 36/886; A61K 36/28; A61K 36/76; A61P 17/02; A61Q 1/14; A01P 1/00; A61K 31/60; C11D 17/00

(52) U.S. Cl. 424/401; 424/443; 424/405; 424/744; 424/764; 424/771; 514/159; 510/445

(57) ABSTRACT

An object made of at least two layers is disclosed, in which a first layer is a biologically degradable water-soluble support material containing an active agent and connected to at least one second layer, which is made up of a biologically degradable but water-insoluble material in the form of fibers. The active agents can be pharmaceutical agents, cosmetics, cleaning agents, agrochemicals and biocides. The object serves for the controlled release of at least one active agent based on the effect of water. The fiber structure of the second layer permits a control of the speed of water entry and/or exit.

The present invention relates to an object made of at least two, preferably three, layers. A first layer comprises an active agent and is preferably water-soluble. The second layer is composed of water-insoluble fibers. The fiber structure permits control of the rate of water entry and/or exit and consequently also control of the release of the active agent from the first layer.

The range of objects which release an active agent in a controlled manner is very extensive. These are objects which—as soon as the active agent has been more or less completely released—can be discarded. The disadvantage associated with this is the need for disposal of these spent objects and their dumping in landfill.

It is an object of the present invention to provide an object which is capable of releasing an active agent in a controlled manner into a surrounding area and in which, following application as intended, the remaining constituents are 100% biologically degradable.

It is a particular object of the present invention to provide an object which is able, upon contact with water, to release the active agent without significant degradation of the constituents of the object taking place during this time.

The object is achieved by an object made of at least two layers.

The first layer comprises a support material and at least one active agent. It is swellable or erodible in water or, as constitutes a preferred embodiment, soluble in water. Suitable support materials for the first layer are biologically degradable materials which are also swellable, erodible and/or soluble in water.

The second layer is composed of a water-insoluble, but biologically degradable material which is in the form of fibers. The fiber structure can bring about control of the rate of water entry and/or exit. In this way, the second layer can also exert control of the release of the active agent from the first layer. In one particular embodiment, the second layer can be firmly joined to the first layer.

In a further preferred embodiment, the object (in the form of a “multilayer system”) comprises a third layer which—like the second layer—is composed of a water-insoluble, but biologically degradable material. This third layer can likewise be in the form of fibers, although other forms are also possible. For example, this layer can also be executed in the form of a film.

In a preferred embodiment, the second layer and the third layer are joined together and completely enclose the first layer. For this, these two layers have a larger area than the first layer and on all sides of the object extend beyond the first layer.

A method of producing an object or a multilayer system made of at least one first, active-agent-containing layer and at least one second fiber layer is further provided by the invention.

The use of an object or of a multilayer system made of at least one first, active-agent-containing layer and at least one second fiber layer for the controlled release of at least one active agent is further provided by the invention. Here, the release of the at least one active agent takes place due to the action of water on the multilayer system. Active agents to be mentioned are in particular pharmaceutical active agents, cosmetics, cleaning agents, agrochemicals and biocides.

The object is composed of at least two layers, which is to be understood as meaning that it comprises at least two sheet-like and superimposed material masses.

The first layer comprises at least one active agent and a support material. The first layer is swellable in water, erodible or soluble in water. A water-soluble embodiment of the first layer is preferred.

Suitable active agents are in particular pharmaceutical active agents, cosmetics, cleaning agents, agrochemicals and biocides.

Pharmaceutical active agents (drugs or chemotherapeutics) are active agents and remedies. These substances are known to the person skilled in the art from relevant sources, for example the German pharmacopeia or the “Red List”, the Yellow List Pharmaindex and similar indexes. Preference is given in particular to those pharmaceutical active agents which can be used externally. These include pharmaceutical active agents which are used, for example, for skin diseases (dermatotherapeutics) or for wound healing. Preferably, of suitability are therefore antibiotics, antiallergics, disinfectants, antihistamines, antiscabies agents, corticoids, antipruritics, tar preparations, psoralens, retinoids, photoprotective substances, keratolytic and caustic drugs, anti-inflammatories, antipsoriasis agents, antibacterial active agents, antiviral active agents, fungicidal active agents (antimycotics), surface anesthetics and steroids.

Cosmetics are substances or preparations made of substances which are intended exclusively or predominantly to be used externally on the human body or in its oral cavity for cleaning, care, protection, maintaining a good condition, perfuming, changing the appearance or for influencing body odor. Cosmetics include, in particular, substances for skincare such as bath preparations, skin washing and cleaning agents, skincare agents, eye cosmetics, lip care agents, nail care agents, intimate care agents and foot care agents; substances with a specific effect such as photoprotective agents, skin tanning agents, depigmentation agents, deodorants, antihydrotics, hair removal agents, shaving agents and fragrances; substances for dental and oral care such as dental and oral care agents, denture care agents and denture adhesives and also substances for hair care such as hair washing agents, hair care agents, hair strengthening agents, hair shaping agents and hair colorants. Information on cosmetic active agents and auxiliaries which are used in the formulation of cosmetic products can be found by the person skilled in the art in the “Kosmetikjahrbuch [Cosmetics yearbook]” published by the publishers for the chemical industry H. Ziolkowsky GmbH, Augsburg, and also the “International Cosmetic Ingredient Dictionary and Handbook” and the “CTFA International Buyers' Guide”, which are both published by The Cosmetic, Toiletry, and Fragrance Association, Washington. A further handbook is H. P. Fiedler: “Lexikon der Hilfsstoffe far Pharmazie, Kosmetik and angrenzende Gebiete [Lexicon of auxiliaries for pharmacy, cosmetics and related fields]”, Editio Cantor-Verlag, Aulendorf (1996). Reference is made to these works, which appear regularly at specific intervals, in their entirety, especially as regards the classification of the substances as regard to their function (intended use) and the nomenclature of the substances in question.

Cleaning agents are in particular surfactant-containing formulations with a very wide field of use and very differing composition dependent thereon. The most important groups are household cleaners, industrial (technical) and institutional (commercial) cleaners. A distinction is made between alkaline, neutral and acidic cleaning agents according to the pH. The cleaning agents include all-purpose cleaners and special cleaning agents such as automobile care agents, oven cleaners, deliming agents, window cleaners, stain removal agents, floor care agents, glass ceramic hob cleaners, hearth care agents, leather care agents, metal polishes, furniture care agents, pipe cleaning agents, sanitary cleaners, scouring agents, carpet care agents and WC cleaners.

The main constituent of cleaning agents is surfactants. The person skilled in the art knows of these interface-active substances on account of their ability to reduce the interfacial tension. Surfactants are amphiphilic (bifunctional) compounds with at least one hydrophobic and one hydrophilic molecular moiety. The hydrophobic radical is in most cases a hydrocarbon chain having 8 to 22 carbon atoms which is as linear as possible. Specific surfactants also have (dimethyl)siloxane chains (silicon surfactants) or perfluorinated hydrocarbon chains (fluorine surfactants) as hydrophobic molecular moiety. The hydrophilic radical is either a negatively or positively electrically charged (hydratable) or a neutral polar head group. Interface-active betaines or amino acid surfactants (amphoteric or zwitterionic surfactants) carry negatively and positively charged groups in one molecule. Base properties of the surfactants are the oriented adsorption onto interfaces and the aggregation to give micelles and the formation of lyotropic phases.

Surfactants are divided according to the nature of their hydrophilic head groups:

| Class | Hydrophilic group | Typical representatives |
|---|---|---|
| Anionic | —COO⁻ | soaps |
| surfactants | —SO₃— | alkylbenzenesulfonates, alkanesulfonates |
| | —OSO₃— | alkyl sulfate (e.g. fatty alcohol sulfates) |
| | —(CH₂—CH₂—O)ₓ—SO₃— | alkyl ether sulfates |
| | (x = 1 to 4) | |
| Nonionic | —(CH₂—CH₂—O)ₓ— | fatty alcohol polyglycol ethers, alkylphenol polyglycol ethers |
| surfactants | (x = 2 to 20) (opt. | (APEO), (ethoxylated) sorbitan fatty acid esters, alkyl |
| | modified) sugars | polyglucosides (APG), fatty acid glucamides, fatty acid |
| | | ethoxylates, ethylene oxide-propylene oxide block polymers, |
| | | polyglycerol fatty acid esters, fatty acid alkanolamides |
| | 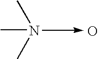 | amine oxides |
| Cationic surfactants | 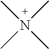 | quaternary ammonium compounds with one or two hydrophobic groups (e.g. cetyltrimethylammonium bromide and cetyltrimethylammonium chloride); salts of long-chain primary amines |
| | 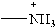 | |
| Amphoteric surfactants | 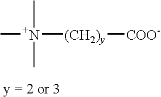 | N-(acylamidoalkyl) betaines (e.g. cocoamidopropylbetaine), N-alkyl-β-aminopropionates and N-alkyl-β-iminoproprionates |
| Block copolymers | (CH₂CH₂O)ₓ-[(EO)ₓ] in | low-foam surfactants |
| | (EO)ₓ(PO)_{y}(EO)ₓ | |
| | (x, y > 10) | |
| | (EO = ethylene oxide, | |
| | PO = propylene oxide) | |
| Polyelectrolytes | | chelating agents, flocculants |

The most important (anionic) surfactants include soaps, linear alkylbenzenesulfonates (LAS), fatty alkyl polyethylene glycol ether sulfates (FAES), such as, for example, sodium lauryl ether sulfate, fatty alcohol sulfates (AS, FAS), the most important (nonionic) surfactants include fatty alcohol polyglycol ethers (fatty alcohol ethoxylates, FAEO) and alkylphenol polyglycol ethers (APED).

Agrochemicals and biocides are chemicals which are used in agriculture, horticulture and domestically, for example fertilizers, herbicides, fungicides, insecticides and other crop protection agents and pest control agents, repellants, attractants, plant treatment agents, storage protection agents, plant growth agents and plant inhibitors, silaging agents and preservatives and also soil improvers. The person skilled in the art is aware of these substances, for example, from “The Pesticide Manual”, 9th edition, published by The British Crop Protection Council, (1991) or the “List of approved crop protection agents”, which is published at certain intervals by the Federal Office for Consumer Protection and Food Safety.

The active agent is dissolved or dispersed in the support material. The active agent does not have to be water-soluble but water-soluble active agents are preferred. The amount of active agent present in the object is essentially dependent on the particular intended use. An individual object can be loaded with up to 90% by weight of active agent. Preference is given to active agent loadings between 40 and 70% by weight. The content of active agent may naturally also be lower, particularly in the case of highly effective active agents.

Suitable support materials for the first layer are biologically degradable materials which are swellable, erodible and/or soluble in water. These materials are polymeric materials which are of natural origin or are synthetically produced.

Biologically degradable is understood as meaning that the material in question can be degraded by microorganisms into natural metabolic products in a biologically active environment (compost, digested sludge, earth, wastewater). The degree of biological degradability is determined by measuring the degradation in an aerobic or anaerobic medium. In an aerobic medium, the CO₂ formation and/or the O₂ depletion is determined, in the anaerobic medium the CH₄ formation and/or the CO₂ formation is used. These measurement methods are known to the person skilled in the art from the OECD tests 301 A-F or corresponding equivalent methods. According to these, these materials are “completely biologically degradable” if, in a 28-day test under aerobic conditions, more than 60% of the theoretical maximum value of the biological degradability is achieved on account of the O₂ consumption and/or the CO₂ formation.

Within the context of the present invention, in particular also the synthetic materials which meet the criteria of the harmonized EN standard EN 13432 (“Demonstration of the compostiblity of synthetic products”), are to be understood as “biologically degradable”.

Within the context of this description, “soluble in water” means that complete hydration takes place and a solution is formed, i.e. a homogeneous mixture of water as solvent and the support material and/or the active agent as “dissolved material”.

The term “swellable in water” means that, upon contact with water, water molecules penetrate into the biologically degradable material and these bring about a change in volume and shape and form a gel. In contrast to unrestricted swelling during which ultimately the swelling substance converts to a solution or suspension, within the context of this description, the term “swellable in water” is to be understood as meaning restricted swelling during which the gel which is formed remains coherent.

The term “erodible in water” is to be understood as meaning that the biologically degradable material can disintegrate, upon contact with water, into smaller units or segments. These can be easily separated off mechanically, e.g. rinsed away with water. In this regard, it is not necessary for the material to be completely soluble in water or swellable in water. A practical guide for the property “erodible” is the detail that after composting for 3 months and subsequent sieving through a 2 mm sieve, not more than 10% residues, based on the original mass, must be left over.

“Biologically degradable materials which are soluble in water” include water-soluble cellulose, cellulose derivatives and polyvinyl alcohol (PVA) and polyvinylpyrrolidone (PVP), copolymers of PVA and PVP and also pullulan. Hydroxypropylcellulose (HPC) and hydroxypropylmethylcellulose (HPMC) are preferred cellulose derivatives, where their dissolution behavior in water also depends on their degree of polymerization and any possible crosslinking. Thus, these polymers dissolve more rapidly in water if they have a relatively low degree of polymerization.

“Biologically degradable materials which are swellable in water” include cellulose which is swellable in water, starch, gelatin, galactomannans, water-swellable cellulose products, tragacanth, polyglycosides, polyamides, polyacrylamide, carboxyvinyl polymers, agar-like algae products, mixed polymers of methyl vinyl ether and maleic anhydride, guar gum, hydroxypropylguar gum, guar flour, gum arabic, dextrin, dextran, microbiologically obtained polysaccharide gums, synthetically obtained polysaccharides, methyl glucose derivatives, hydroxymethylpropylcellulose, polygalacturonic acid derivatives, pectin and pectin amide.

The first layer can be in the form of a compact layer, for example as film. It may also be in the form of a solid foam with air bubbles or as nonwoven fabric. Preference is given to the film form.

Preferably, the first layer is flexible.

The dimensions of the first layer are governed primarily by the desired intended uses. This means that the dimensions are selected according to the required amount of active agent and the site of use. There are thus in practice no technical restrictions for the choice of dimensions. However, since the objects should also have the most advantageous dimensions possible for handling, the following dimensions are preferred: the first layer can have a thickness between 12 μm and 5 mm, preferably between 50 μm and 2 mm and particularly preferably between 70 μm and 180 μm. The width here can be between 5 mm and 30 cm, preferably between 2 cm and 12 cm. These dimensions are also valid for the length. It goes without saying here that in the case of a square or round first layer, these values are equal. Otherwise, the terms should be interpreted such that the length is greater than the width and these two expanses are always larger than the thickness.

If a surfactant is present as active agent in the first layer, this layer can have an areal weight of from 40 to 60 g/m², preferably 55 to 60 g/m². In cases where an oil (i.e. a lipophilic active agent, i.e. a vegetable or animal fatty oil which consists essentially of mixed triglycerides of higher fatty acids) is present in the first layer, the latter can assume an areal weight of from 200 to 210 g/m².

The second layer of the object is water-insoluble, but impermeable to water. This layer is likewise composed of a biologically degradable material.

Within the context of this description “water-insoluble” means that the second layer neither “erodes in water” nor forms a solution upon contact with water.

The term “permeable to water” is to be understood as meaning that molecules of water can pass in liquid and also in gaseous form through the second layer.

“Biologically degradable materials which are insoluble in water” include polylactic acid, aliphatic polyesters (e.g. Bionelle®), polycaprolactone, polypropiolactone, polyhydroxybutyrate, polyhydroxyvalerate, polyglycolic acid, polysaccharides (chitin), chitosan, polypeptides and collagen and also copolymers of the corresponding hydroxy acids. Preference is given to polylactic acid and polyglycolic acid.

So that the second layer is permeable to water, the biologically degradable and water-insoluble material is in the form of fibers which are joined together such that a sheet material is formed. This sheet material can on the one hand be a nonwoven fabric, on the other hand a woven or knitted fabric.

One particular embodiment constitutes a material of polylactate and cellulose, where the fraction of these two components can vary between 30-100% by weight of polylactate and 0-70% by weight of cellulose. In this connection, in a sheet material produced from the fibers of these two materials—preferably a nonwoven—the concentration of one of these two components can vary. Thus, in one such nonwoven, the upper side can be formed completely of polylactate whereas on its underside it is composed of a mixture of cellulose and polylactate. This “internal concentration gradient” can influence the properties of the corresponding nonwoven—in particular with regard to its permeability to water and its ability to be welded.

The fibers are in principle continuous material strands. Whereas the length and the diameter of the fibers of biologically degradable materials of natural origin are restricted according to their origin (e.g.: wool, silk, cotton), the length, diameter and in particular the fiber cross section of fibers of synthetically biologically degradable materials can vary according to their production method. Thus, in the case of these fibers, diameters of less than 12 μm are preferred.

Since as a rule individual fibers are spun from round spinning nozzle holes in the wet or dry spinning method, during consolidation, the fiber cross section can assume various forms, i.e. besides a round shape, also bean- or kidney-shaped, but also jagged, triangular, rectangular etc. Further modifications of the original shapes can be achieved later by means of refining processes. During the melt-spinning, it is possible to modify the fiber cross section in a targeted manner by using different profile spinning nozzles, e.g. triangular, three-lobed or stellate, as a result of which the fibers gain a structured and greatly increased surface relative to the volume. With regard to shine, color effect, elasticity and feel, these profile fibers have considerably improved properties. By using specially shaped nozzles or cavities spun into the inside of the fibers with the inclusion of air, the hollow fibers are produced.

The fiber structure of the second layer contributes to the water being able to penetrate and pass through the second layer in a controlled manner. This is possible due to the fibers being processed to give wovens or nonwovens. A woven is understood as meaning a (rectangular at least during the production) sheet material composed of threads in the longitudinal direction (warp threads) and cross threads (weft threads). Nonwovens differ, by contrast, from wovens by a positioning of the individual fibers or threads determined by the production method. Nonwovens consist of fibers whose position can only be described using the methods of statistics. The fibers are present in the nonwoven in a random nature relative to one another.

Random-fiber nonwovens of this type are produced in the dry or wet state using various processes. The dry preparation takes place in a stream of air, which is often supported by electrostatic charging in order to achieve uniform fiber distribution. The wet preparation can take place in water. Randomizing or condenser rolling on a roller card is also known. The compactness, i.e. the density of the fabric threads or the fibers of the nonwoven of the second layer then essentially determines the rate of water entry and/or exit in this layer.

The second layer is preferably free from an active agent. However, it is permeable to the active agent.

The first layer and the second layer can be firmly joined together. For this, for example, the first layer can be adhesively finished by adding an adhesive. However, the two layers can also be joined with the help of an additional adhesive layer positioned between first and second layer. In both cases, the adhesive used is preferably a biologically degradable adhesive. Biologically degradable adhesives include, for example, latex (the milky sap of the rubber tree). The sticking of the first and second layer can be over the entire surface; however, it may also take place only at points or (if appropriate only in sections) along the outlines of the two layers or of the smaller layer in terms of area.

In a particularly preferred embodiment, the object is in the form of three layers, the first layer being situated between the second layer and a third layer or enclosed by both of these (as “multilayer system”). In this connection, in a particular embodiment, the third layer can be identical to the second layer. In this case, it is also possible for the second layer to completely enclose the first, which can be accomplished in terms of production as in the case of the known forming, filling and sealing methods using packaging machines with which tubular bags are produced in this way.

As the third layer it is also possible to use a water-impermeable layer of a biologically degradable material. For this, the biologically degradable material can be in the form of a film.

In a further embodiment, the object can comprise a fourth layer, preferably in the form of a high-loft nonwoven. A high-loft nonwoven is understood by the person skilled in the art as meaning a nonwoven which is very light in weight for a comparatively large volume. The thickness of such a nonwoven layer can be in the range from 1 to 8 cm with an areal weight of from 50 to 800 g/m². This fourth layer can be positioned between the first and the second layer, but also above or below the laminate of first and second layer. It can—if the object has a third layer—also be arranged between the first and the third layer.

In a particular embodiment, the object comprises a residual moisture of less than 5% by weight, preferably less than 2% by weight. The object as a whole is flexible, which can be attributed to the flexibility of the individual layers.

The object can comprise preservatives which prolong the durability against the effect of microorganisms. Such preservatives include certain biocides, germination preventers, but also heavy metal salts, organic acids such as salicylic acid, benzoic acid, sorbic acid, propionic acid, hydroxysuccinic acid, propionic acid, boric acid, formic acid, fumaric acid and other acids and also the salts of these acids, but also phenoxyethanol, diazolidinylurea and 4-hydroxybenzoic acid and esters of 4-hydroxybenzoic acid. In a particular embodiment, however, the object is free from preservatives, in particular it is free from phenoxyethanol, 4-hydroxybenozic acid and/or esters of 4-hydroxybenzoic acid (parabens). The absence of preservatives has the advantage that they do not stand in the way of composting, i.e. the biological degradability of the spent object.

During its use, the object comes into contact with water. This enters through the second layer and comes into contact with the first layer. According to the properties of the support material (i.e. depending on the swellability, erodibility or solubility), the process of dissolution of the first layer starts and so too does the controlled release of the active agent.

Besides the active agent and the support material, the first layer can comprise further components, such as, for example, fillers, dyes, fragrances, flavorings, emulsifiers, antioxidants, pigments and similar auxiliaries which are known to the person skilled in the art from the handbooks specified above. The basic formulations below list further such substances with their function. Substances of this type may also be present in the second and/or third layer.

Consolidation of the nonwoven can take place thermally, by needle bonding and also rubber bonded.

Possible areas of application for the object depend on the choice of active agents: medical indications, cosmetic treatments, cleaning and disinfection in the home, hospitals and industry, and also administration of crop protection agents are contemplated. Preferably, for this, the object is brought into contact with an adequate amount of water, which sets in motion the dissolution of the first layer and permits release of the active agent. As a result of the dissolution behavior of the first layer, it is possible to control the rate of release of the active agent. A first layer which dissolves only slowly means that the object releases the active agent at a relatively constant rate over a long period.

After the object has come into contact with an adequate amount of water, it can be brought into contact with the article/surface to be treated. In this way, it is ensured that the active agent can act at the desired site (for example: skin, mucosa, clothing, furnishings, floorings, window panes, car bodies, dishes, agriculturally used soil etc.).

The first layer and the second layer are preferably produced in two separate working steps and then joined together. This can take place by sticking the first and second layer. In the case of an object composed of three layers, the first layer can, however, also be placed onto a second layer such that the second protrudes at the side and after covering with a third layer, which likewise protrudes at the side beyond the first layer, binding of the first and third layer takes place. This binding of the areas of the first and third layer protruding at the side can take place, for example, by adhesion, needle bonding, by sewing; by thermowelding, by blowing, by water-jet treatment, by a chemical bond or by needle bonding. A particular embodiment of a method for joining the layers is ultrasonic welding.

The invention will be described in more detail by the examples below.

### EXAMPLE 1

### General Description for the Production of an Object with an Oil as Active Agent (“Oil Pad”) for Cosmetic Applications

To produce layer 1, an emulsifier or a mixture of two or more emulsifiers, glycerol and water are initially introduced and, at 75° C., an oil (i.e. an active agent with lipophilic character) or a mixture of different oils is slowly added with stirring. The mass which forms is cooled to 40° C. with stirring and finally perfume is incorporated.

In a second vessel, hydroxypropylcellulose and/or hydroxypropylmethylcellulose are dissolved in water with vigorous stirring. 4% strength aqueous carrageen solution, corn starch and the oil mixture prepared previously are added and the mixture is left to foam for 20 minutes at maximum stirring speed. The resulting mass is coated in a coating box with a thickness of 1000 μm onto the siliconized side of a paper support coated with polyethylene and dried at 70° C. for ca. 90 minutes in the laboratory drying cabinet.

The resulting dry foam (layer 1) is cut, removed from the support material and placed in the center of a piece of nonwoven the edges of which are ca. 1 cm longer (layer 2). This is covered with a further layer of nonwoven of the same size (layer 3) and the two outer layers are joined by thermowelding.

### EXAMPLE 2

### Preparation of an Object with a Surfactant for Cleaning Purposes

To produce layer 1, 9.53 g of a surfactant-containing cleaning mixture (Desinol PG; a commercially available surfactant mixture) and the same amount of caprylyl/capryl glucoside (CCG) are added with vigorous stirring to 52 g of a 25% aqueous polyvinyl alcohol solution (Mowiol 8-88). 0.19 g of perfume and 0.06 g of sodium salicylate and 0.134 g of sodium benzoate are then added and the mixture is stirred to optical homogeneity. The resulting foamy mass is coated in a coating box with a thickness of 300 μm on the siliconized side of a paper support coated with polyethylene and dried at 70° C. for ca. 20 minutes in a laboratory drying cabinet.

The resulting dry foam (layer 1) is cut into square sections with edge length of 3 cm, removed from the support material and placed in the middle of a piece of nonwoven (made of polylactate/cellulose in the mixing ratio 35:65) whose edges are ca. 1 cm larger (layer 2). This is covered with a further layer of nonwoven of the same size (layer 3) and the two outer layers are joined by thermowelding.

For use, the object consisting of three layers (“surfactant pad”) is treated with ca. 10 ml of water by immersing into water, and activated by gently kneading in the hand, which makes it easier for the surfactant to leave the object. By gently pressing—for example on the facial skin—the effect of the surfactant on the skin is made possible.

For the various applications, objects can be produced which are in each case described by the following general basic formulations. The tables specify substances and groups of substances with their function and also the quantitative fractions in % by weight (in the solid end product, i.e. the first layer).

Basic formulation for alkaline floor cleaner

| Ingredients | Function | % |
|---|---|---|
| Fatty alcohol polyglycol | cleaning, antistatic | 5.5-32.5 |
| ether phosphate, K salt | | |
| Fatty alcohol ethoxylated/ | cleaning | 2.75-32.5 |
| propoxylated | | |
| Fatty acid amidoamine | hydrophobicization | 0.55-13.0 |
| Organic acid, Na salt | dispersion | 0.55-6.5 |
| Monoethanolamine | alkali, cleaning | 0-3.25 |
| | booster | |
| Triethanolamine | alkali, cleaning | 0-3.25 |
| | booster | |
| Methoxymethylethoxypropanol | solubility promoter | 2.75-13 |
| PVA | film former | 35-45 |

Basic formulation for ceran polish

| Ingredients | Function | % |
|---|---|---|
| Aminoorganopolysiloxane | hydrophobicization | 2.75-19.5 |
| Cyclomethicones | shine imparter | 0.55-13 |
| Silicone oil | hydrophobicization | 2.75-19.5 |
| Abrasives | cleaning | 2.75-26 |
| Fragrance | fragrance | 0-1.625 |
| Preservative | preservation | 0-0.325 |
| PVA | film former | 35-45 |

Basic formulation for automobile polish

| Ingredients | Function | % |
|---|---|---|
| Propylene glycol | lubricant | 2.75-32.5 |
| Quaternary silicone | hydrophobicization | 0.55-6.5 |
| compound | | |
| Cyclomethicones | shine imparter | 2.75-13 |
| Fatty acid diethanolamide | wetter | 0.55-6.5 |
| C8-C18 | | |
| Polyether siloxane | hydrophobicization | 0.55-6.5 |
| Fragrance | fragrance | 0-1.625 |
| Preservative | preservation | 0-0.325 |
| PVA | film former | 35-45 |

Basic formulation for floor wiping care

| Ingredients | Function | % |
|---|---|---|
| Acrylic polymer | shine imparter | 0.55-13 |
| Dimethicones | hydrophobicization | 5.5-32.5 |
| Fatty alcohol ethoxylate | cleaner | 5.5-19.5 |
| C8-C18/2-20 EO | | |
| Monoethanolamine | alkali, cleaning | 0.55-0.13 |
| | booster | |
| Citric acid | pH extender | 0.055-0.13 |
| Fragrance | fragrance | 0-1.625 |
| Preservative | preservation | 0-0.325 |
| PVA | film former | 35-45 |

Basic formulation for dishwashing compositions

| Ingredients | Function | % |
|---|---|---|
| Alkanesulfonate | cleaner | 5.5-32.5 |
| Sodium lauryl ether | cleaner | 5.5-32.5 |
| sulfate C12/2-4 EO | | |
| Fatty alcohol ethoxylate | cleaner | 0.275-6.5 |
| C8-C18/2-20 EO | | |
| Alkyl ether carboxylic | wetting agent | 0.055-3.25 |
| acid C8/6-8 EO | | |
| Sulfosuccinic acid ester | wetting agent | 0.00055-0.065 |
| C8-C12, Na salt | | |
| Fatty acid | foam stabilizer | 0.55-6.5 |
| diethanolamide C8-C18 | | |
| Fragrance | fragrance | 0-1.625 |
| Preservative | preservation | 0-0.325 |
| PVA | film former | 35-45 |

Basic formulation for power cleaner

| Ingredients | Function | % |
|---|---|---|
| Alkanesulfonate | cleaning | 5.5-32.5 |
| Sodium lauryl ether | cleaning | 0.55-13 |
| sulfate C12/2-4 EO | | |
| Fatty alcohol ethoxylate | cleaning | 2.75-19.5 |
| C8-C18/2-20 EO | | |
| Fatty acid alkali metal | cleaning | 0.55-6.5 |
| salt C8-C18 | | |
| Fragrance | fragrance | 0-1.625 |
| Sodium citrate | stabilizer | 0-0.975 |
| PVA | film former | 35-45 |

Basic formulation for plastics cleaner

| Ingredients | Function | % |
|---|---|---|
| Dipropylene glycol | antistatic | 0.55-13 |
| monobutyl ether | | |
| Fatty alcohol polyglycol | cleaning, | 0.55-13 |
| ether phosphate, K salt | antistatic | |
| Dimethicones | spreading agent | 5.5-32.5 |
| Fatty alcohol ethoxylate | cleaning, | 5.5-19.5 |
| C8-C18/2-20 EO | antistatic | |
| Na salt of organic acids | complexing agent | 0.55-6.5 |
| Fragrance | fragrance | 0-1.625 |
| Preservative | preservation | 0-0.325 |
| PVA | film former | 35-45 |

Basic formulation for furniture care

| Ingredients | Function | % |
|---|---|---|
| Carnauba wax | care | 0.55-13 |
| Beeswax | care | 0.55-13 |
| Shellac wax | hydrophobicization | 0.55-13 |
| Orange oil (limonene) | solubility promoter | 5.5-52 |
| PVA | film former | 35-45 |

Basic formulation for neutral cleaner

| Ingredients | Function | % |
|---|---|---|
| Alkanesulfonate | cleaner | 5.5-32.5 |
| Sodium lauryl ether | cleaner | 0.55-19.5 |
| sulfate C12/2-4 EO | | |
| Fatty alcohol ethoxylate | cleaner | 2.75-32.5 |
| C8-C18/2-20 EO | | |
| Alkyl ether carboxylic | wetting agent | 0.055-0.325 |
| acid ester C8/6-8 EO | | |
| Sulfosuccinic acid ester | wetting agent | 0.00055-0.65 |
| C8-C12, Na salt | | |
| Fragrance | fragrance | 0-1.625 |
| Preservative | preservation | 0-0.325 |
| PVA | film former | 35-45 |

Basic formulation for silver polish

| Ingredients | Function | % |
|---|---|---|
| Sodium dithionite | bleach | 2.75-58.5 |
| Sodium carbonate | alkali | 2.75-16.25 |
| Sodium hydrogencarbonate | diluent | 0.55-32.5 |
| Nitrilotriacetic acid | complexing agent | 0.55-6.5 |
| PVA | film former | 35-45 |

Basic formulation for carpet cleaner

| Ingredients | Function | % |
|---|---|---|
| Sodium lauryl ether sulfate | cleaner | 0.55-19.5 |
| C12/2-4 EO | | |
| Sodium fatty alcohol sulfate | foam stabilizer | 0.55-19.5 |
| C12-C18 | | |
| Fatty acid amidoalkylbetaine | cleaner | 0.55-19.5 |
| C12-C14 | | |
| Fatty acid diethanolamide | foam stabilizer | 0.55-6.5 |
| C16-C18 | | |
| Sulfosuccinic acid ester | wetting agent | 0.00055-0.65 |
| C8-C12, Na salt | | |
| Sheet silicate; alkali metal | imparting | 0.005-3.25 |
| salt | thixotropy | |
| Fragrance | fragrance | 0-0.65 |
| Preservative | preservation | 0-0.325 |
| PVA | film former | 35-45 |

Basic formulation for makeup removal emulsion

| Ingredients | Function | % |
|---|---|---|
| Triglyceride of vegetable fatty | skincare, | 11.0-26 |
| acids C6-C14 | makeup removal | |
| Glycerol | humectant | 2.75-6.5 |
| Sugar fatty acid ester C16 | emulsion | 2.75-6.5 |
| | formation | |
| Branched fatty acid ester C6-C12 | skincare, | 2.75-6.5 |
| | makeup removal | |
| Sugar fatty acid ester C12 | emulsion | 0.55-6.5 |
| | formation | |
| Branched fatty acid ester | skincare, | 0.55-3.25 |
| C14-C18 | makeup removal | |
| Fragrance | fragrance | 0-0.65 |
| Support material | film former | 35-45 |
| (hydroxypropylmethylcellulose, | | |
| carrageenan, | | |
| hydroxypropylcellulose) | | |

Basic formulation for hand cleaning foam/washing lotion

| Ingredients | Function | % |
|---|---|---|
| Monoisopropanol fatty alcohol | cleaning | 0-39 |
| ether sulfate C12-C14/2 EO | | |
| Sodium lauryl ether sulfate | cleaning | 0-39 |
| C12/2-4 EO | | |
| Fatty acid glyceryl ester, | refatting | 2.75-13 |
| ethoxylated C8-C18 | agent | |
| Fatty acid amidoalkylbetaine | foam | 0.55-6.5 |
| C12-C14 | stabilizer | |
| Fatty acid diethanolamide C8-C18 | foam | 0.55-6.5 |
| | stabilizer | |
| Sodium lauryl sulfate C12 | foam | 0-1.3 |
| | stabilizer | |
| Fragrance | fragrance | 0-1.3 |
| PVA | film former | 35-45 |

Basic formulation for face cleaning foam, oil-
containing

| Ingredients | Function | % |
|---|---|---|
| Polyglyceryl ester C8-C18 | cleaning | 2.75-13 |
| Glycerol | humectant | 13.75-25 |
| Triglyceride of vegetable fatty | skincare, | 16.5-39 |
| acids C6-C14 | makeup removal | |
| Branched fatty acid ester | skincare, | 0.55-6.5 |
| C6-C12 | makeup removal | |
| Branched fatty acid ester | skincare, | 0.55-6.5 |
| C14-C18 | makeup removal | |
| Fragrance | fragrance | 0-1.3 |
| PVA | film former | 35-45 |

Basic formulation for skin clarifying foam

| Ingredients | Function | % |
|---|---|---|
| Polyglyceryl ester C8-C18 | cleaning; refatting | 5.5-26 |
| | agent | |
| Glycerol | humectant | 5.5-19.5 |
| Plant extracts (e.g. aloe | moisturizer | 0.0275-2.75 |
| vera, larch, cucumber) | | |
| Dead sea salt | flake detachment | 0.55-3.25 |
| Plant extract (e.g. aloe | inflammation | 0.0275-1.3 |
| vera, chamomile, | inhibition | |
| hamamelis, marigold) | | |
| Plant extract (e.g. birch, | wound healing | 0.0275-1.3 |
| willow bark, Gotu Kola) | promotion, scar | |
| | formation reduction | |
| Plant extract (e.g. | reduction in sebum | 0.0275-1.3 |
| licorice, green tea) | production | |
| Salicylic acid | flake detachment | 0.0275-1.3 |
| Allantoin | skin calming | 0.055-0.65 |
| Fragrance | fragrance | 0-1.3 |
| PVA | film former | 35-45 |

Basic formulation for face tonic

| Ingredients | Function | % |
|---|---|---|
| Propylene glycol | humectant | 0-32.5 |
| Glycerol | humectant | 0-32.5 |
| Silicone surfactant | wetting | 0-13 |
| Silicone oil | conditioning, | 0-6.5 |
| | emollient | |
| Plant extract (e.g. | toning | 0-3.25 |
| hamamelis, horse chestnut, | | |
| butcher's broom) | | |
| Allantoin | skin calming | 0-3.25 |
| Plant extracts (e.g. aloe | moisturizer | 0-3.25 |
| vera, larch, cucumber) | | |
| Protein (silk, plant, | moisturizer, film | 0-3.25 |
| milk, animal, marine) | former | |
| Fragrance | fragrance | 0-1.3 |
| PVA | film former | 35-45 |

Basic formulation for face cleaning foam, oil- and PEG-
containing

| Ingredients | Function | % |
|---|---|---|
| Alkyl glucoside C8-C16 | cleaning | 5.5-26 |
| Sodium lauryl ether | cleaning | 2.75-19.5 |
| sulfate C12/2-4 EO | | |
| Fatty acid | cleaning | 2.75-13 |
| amidoalkylbetaine C12-C14 | | |
| Sulfosuccinic acid ester | cleaning | 2.75-13 |
| fatty alcohol ethoxylate | | |
| Na salt, C12-C14/2-4 EO | | |
| Oil ethoxylate based on | refatting agent | 0.55-6.5 |
| vegetable oil 9-26 EO | | |
| Paraffin oil | cleaning | 0.55-6.5 |
| Triglyceride of vegetable | skincare, makeup | 0.275-3.25 |
| fatty acids C6-C14 | removal | |
| Branched fatty acid ester | skincare, makeup | 0.275-3.25 |
| C6-C12 | removal | |
| Fragrance | fragrance | 0-0.65 |
| PVA | film former | 35-45 |

Basic formulation for makeup removal emulsion 2

| Ingredients | Function | % |
|---|---|---|
| Paraffin oil | makeup removal | 5.5-19.5 |
| Alkyl phosphoric acid | emulsion | 0.55-6.5 |
| ester, ethoxylated C12 | formation | |
| Propylene glycol | humectant | 0.55-6.5 |
| Branched fatty acid ester | spreading agent; | 0.55-3.25 |
| C14-C18 | makeup removal | |
| Triglyceride of vegetable | skincare; makeup | 0.055-1.3 |
| fatty acids C6-C14 | removal | |
| Branched fatty acid ester | skincare; makeup | 0.055-1.3 |
| C6-C12 | removal | |
| Silicone surfactant | wetting | 0.055-1.3 |
| Plant extract (e.g. aloe | moisturizer | 0.055-0.65 |
| vera, mallow, cucumber) | | |
| Vitamin E | antioxidant | 0.055-0.65 |
| Panthenol | skin calming | 0.055-0.65 |
| Acrylic acid polymer, Na | stabilizer | 0.0275-0.13 |
| salt | | |
| Fragrance | fragrance | 0-0.65 |
| PVA | film former | 35-45 |

Basic formulation for footbath

| Ingredients | Function | % |
|---|---|---|
| Hydrogenated castor oil, | solubilizer; | 0-39 |
| ethoxylated 40-60 EO | refatting agent | |
| Polyglyceryl ester C8-C18 | solubilizer; | 0-39 |
| | refatting agent | |
| Oil ethoxylate based on | solubilizer; | 0-39 |
| vegetable oil 9-26 EO | refatting agent | |
| Glycerol | humectant; skin | 5.5-19.5 |
| | softening | |
| Essential oil (e.g. | circulation | 0-6.5 |
| rosemary oil; thyme oil; | promoting | |
| eucalyptus oil; pine | | |
| needle oil; ginger oil) | | |
| Essential oil (e.g. | calming; detensing | 0-6.5 |
| sandalwood; lavender oil; | | |
| juniperberry oil; Melissa | | |
| oil; rose oil) | | |
| Essential oil (e.g. citrus | reviving | 0-6.5 |
| oil; mint oil) | | |
| Essential oil (e.g. sage | perspiration | 0-6.5 |
| oil; spruce needle oil) | inhibition | |
| Urea | horny skin | 0-13 |
| | softening | |
| Salicylic acid | horny skin | 0-2.75 |
| | softening | |
| Dead sea salt | horny skin | 0-2.75 |
| | softening | |
| Salicylic acid ester | inflammation | 0-0.65 |
| | inhibition; | |
| | circulation | |
| | promotion | |
| Menthol | refreshing; | 0-0.65 |
| | circulation | |
| | promotion | |
| Camphor | refreshing; | 0-0.65 |
| | circulation | |
| | promotion | |
| PVA | film former | 35-45 |

The preparation of the first layer according to the basic formulations takes place in accordance with the following general scheme:

The other ingredients are weighed into the film former mixture (for example a 25% strength aqueous solution of polyvinyl alcohol (PVA) or a mixture of a 4% strength carrageenan solution, a 25% strength hydroxypropyl-cellulose solution and a 12.5% strength hydroxy-propylmethylcellulose solution). The total mass is stirred to optical homogeneity. Here, the viscosity of the mass can be adjusted, if desired, by adding water such that it can then be foamed up by vigorous stirring. The foamed-up mass is coated with the help of a coating box at a defined layer thickness onto the siliconized side of a support film made of plastic or paper. The layer thicknesses can be up to 5 mm, preferably up to 2 mm. Particular preference is given to layer thicknesses between 200 and 500 μm.

The layered foam produced in this way is then dried at 70° C. for ca. 20 min. After drying, the foam is for further processing, i.e. covered with the second layer (and if appropriate with a third layer) and bonded thereto, if appropriate. Afterwards, the object is ready for further formulation.

It may be mentioned, merely for the sake of completeness, that the preparation of the first layer in the form of a dry foam is a preferred embodiment, but should not be regarded as a limitation thereto.

1. An object made of at least two layers where
a) a first layer is a biologically degradable support material and contains an active agent and is swellable, erodible or soluble in water, and
b) a second layer is composed of a biologically degradable, water-insoluble material in the form of fibers which is permeable to water.

2. The object as claimed in claim 1, characterized in that the biologically degradable support material of the first layer is of natural origin or is produced synthetically.

3. The object as claimed in claim 1, characterized in that the biologically degradable support material of the first layer is constructed from the group comprising cellulose, cellulose derivatives and polyvinyl alcohol (PVA).

4. The object as claimed in claim 1, characterized in that the first layer is in the form of a compact layer, a solid foam or nonwoven.

5. The object as claimed in claim 1, characterized in that the first layer has a thickness between 12 μm and 5 mm, and a width and also a length between 5 mm and 30 cm.

6. The object as claimed in claim 1, characterized in that the first layer comprises at least one active agent from the group of pharmaceutical active agents, cosmetics, cleaning agents, agrochemicals and biocides.

7. The object as claimed in claim 1, characterized in that the biologically degradable material of the second layer is constructed from the group comprising polylactic acid, aliphatic polyesters, polycaprolactone, polypropiolactone, polyhydroxybutyrate, polyhydroxyvalerate, polyglycolic acid, polysaccharides, chitin, polypeptides, collagen, copolymers of the specified hydroxy acids, preferably polylactic acid and polyglycolic acid.

8. The object as claimed in claim 1, characterized in that the biologically degradable material of the second layer is composed of polylactate and cellulose.

9. The object as claimed in claim 1, characterized in that the second layer is present as a sheet material in the form of a nonwoven.

10. The object as claimed in claim 1, characterized in that the second layer is present as a sheet material in the form of a woven fabric or knitted fabrics.

11. The object as claimed in claim 1, characterized in that it has a third layer such that the first layer lies between the second layer and the third layer and the first layer is enclosed by these two layers.

12. A method of producing an object made of at least two layers, where a first layer is a biologically degradable support material and contains an active agent and is swellable, erodible or soluble in water, and a second layer is composed of a biologically degradable, water-insoluble material in the form of fibers, which is permeable to water, characterized in that the first layer and the second layer are produced in two separate working steps and are then joined together.

13. A method for the controlled release of an active agent which comprises of applying water to the object of claim 1, where the object is further characterized in that a first layer is a biologically degradable support material and comprises at least one active agent from the group of pharmaceutical active agents, cosmetics, cleaning agents, agrochemicals and biocides and is swellable, erodible or soluble in water, and a second layer is composed of a biologically degradable, water-insoluble material in the form of fibers, which is permeable to water.

14. The object of claim 5, characterized in that the first layer has a thickness between between 50 μm and 2 mm and a width and also a length between 2 cm and 12 cm.

15. The object of claim 8, characterized in that the biologically degradable material of the second layer is composed of a fraction of between 30-100% by weight polylactate and 0-70% by weight cellulose.
